(19) United States
(12) Plant Patent Application Publication
(10) Pub. No.: US 2017/0112039 P1
(43) Pub. Date: Apr. 20, 2017

(54) Lachenalia jacq. f. ex murray named "RADIENT"

(50) ;

(71) Applicant: AGRICULTURAL RESEARCH COUNCIL, Pretoria (ZA)

(72) Inventor: Riana KLEYNHANS, Benoni (ZA)

(21) Appl. No.: 14/756,820

(22) Filed: Oct. 19, 2015

(51) Int. Cl. A01H 5/00

(52) U.S. Cl. PLT/263.1

(57) ABSTRACT

A new and distinct form of Lachenalia plant characterized by a bulb plant type having a spreading growth habit of a height of 15-30 centimetres (cm) and width of 10-15 cm having a purple flower with cream tips, and green leave with maroon spots, further characterized in that the spots are fairly pronounced in that it has a good contrast.

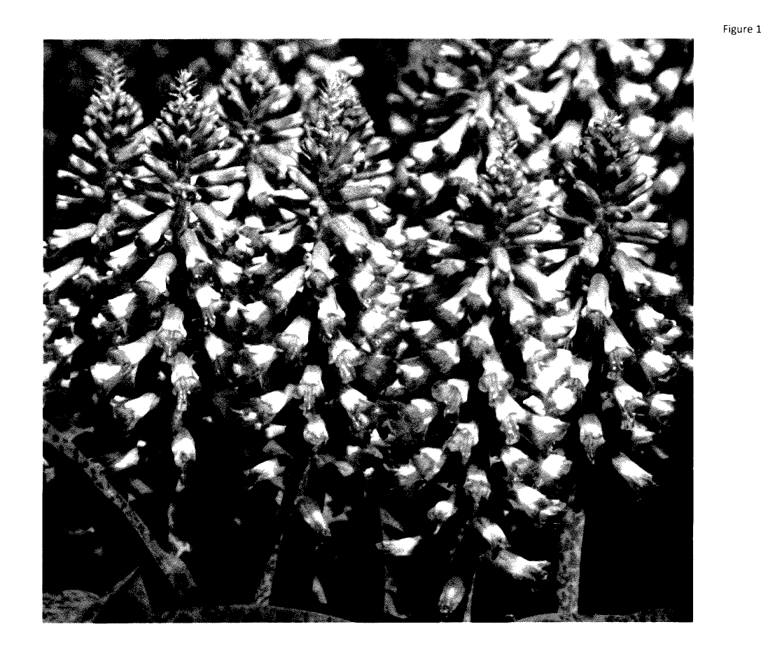

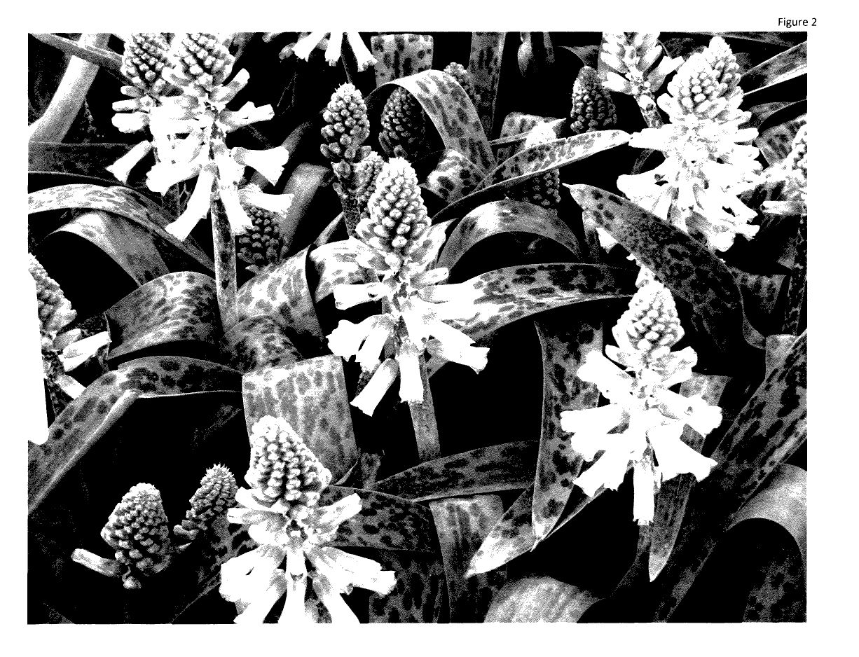

Genus and species: Lachenalia Jacq. f. ex Murray.

Variety denomination: Radient.

### BACKGROUND OF THE INVENTION

The present invention relates to a new and distinct cultivar of Lachenalia, botanically known as Lachenalia Jacq. f. ex Murray having the common name of Viooltjies/Cape cowslip and hereinafter referred to by its variety denomination “Radient”.

The variety was first cultivated in 2008 and originated by means of induced mutation at the premises of Agricultural Research Council Vegetable and Ornamental Plant Institute, Roodeplaat, Pretoria, Republic of South Africa.

Asexual reproduction of the Radient plant has shown that the unique features thereof are stable and reproduced true to type in successive generations.

### SUMMARY OF THE INVENTION

The present invention constitutes a new and distinct variety of Lachenalia which originated from tissue culture as an induced mutation from the variety Rupert as detailed hereinbelow and bred locally.

The following traits have been repeatedly observed and are determined to be the unique characteristics, under the local evaluation conditions, of the Radient plant. These characteristics in combination distinguish Radient as a new and distinct Lachenalia cultivar:
1. Bulb plant type having a spreading growth habit of a height of 15-30 centimetres (cm) and width of 10-15cm.
2. Flower color: purple with cream tips.
3. Leaf color: green with maroon spots.

Plants of the new Lachenalia variety Radient can be compared to the denomination Rupert but differs in that the flower color of Radient is purple with cream tips compared to the purple only of Rupert. The maroon spots of the Radient plant is furthermore more pronounced in that it has a good contrast compared to the low contrast found in Rupert.

### BRIEF DESCRIPTION OF THE PHOTOGRAPHS

The accompanying colored photographs illustrate the overall appearance of the Radient, showing the colors as true as it is reasonably possible to obtain in colored reproductions of this type. The photographs comprise front perspective views of a typical Radient grown in an indoor nursery, but located in an outdoor setting.

### DETAILED DESCRIPTION

The aforementioned photographs, following observations and measurements describe Radient grown by the inventor on behalf of Agricultural Research Council Vegetable and Ornamental Plant Institute, Roodeplaat, Pretoria, Republic of South Africa. Thereafter Radient was made subject of a South African Plant Breeders' Right application in March 2009. This application was granted in 2010 under number ZA20104395.

The plant is a late flowering cultivar and may be used for landscaping and should be planted during the month of March; indoors and in pots only. The plant should be kept at temperatures of 20-25 degrees Celsius during the day and 10-15 degrees Celsius at night. Plenty of light should be provided.

Radient is a mutant derived from the Lachenalia cultivar Rupert through the use of irradiation. Rupert is a hybrid-hybrid cross with the following species used in the hybrid crosses:
Female: [ (L. orchioides subsp glaucina×L. bulbifera)selfed×(L. orchioides subsp glaucina×L. rubida) selfed]; and
Male parent: [(L. aloides var quadricolor×L. rubida)×(L. orchioides subsp glaucina×L. bulbifera)selfed].
Leaf: Monocolored.
   Spots on upper side.—Present and with good contrast.
   Attitude.—Semi-erect.
   Length.—Short to medium — 17.5 cm.
   Width.—Medium to wide — 5.8 cm.
   Ratio length/width.—Large (elongated).
   Shape.—Lanceolate.
   Color.—Dark green.
   Shape in cross section.—Angular.
   Recurving of margin.—Absent.
   Undulation of margin.—Present.
   Blistering and pubescence of upper side.—Absent.
   Spots and color on upper side.—Present (Purplish).
   Size of spots on upper side.—Medium.
   Density of spots on upper side.—Medium.
   Intensity of color of spots on upper side.—Medium.
   Markings on outer side of leaf base.—Present.
Peduncle:
   Length.—Short — 7.5 cm.
   Spots or markings.—Present.
   Size of spots or markings.—Medium.
   Density of spots or markings.—Medium.
Interflorescense:
   Length.—Medium — 15.7 cm.
   Rudimentary apex.—Conspicuous.
   Number of flowers.—Many.
Flower:
   Predominant color.—Purple/Violet (RHS # 31A).
   Intensity of predominant color.—Medium to strong.
   Attitude.—Horizontal.
   Pedicel.—Present — short to medium length.
   Length.—Medium — 2.5 cm.
   Diameter excluding apex.—Medium — 0.7 cm.
   Diameter at apex.—Medium to large — 1.7 cm.
   Attitude of distal part of inner perianth segments.—Spreading.
   Glossiness.—Absent.
   Outer perianth segment.—color of basal part just before opening of flower Violet-blue (RHS # 31A).
   Outer perianth segment.—color of basal part of fully opened flower Violet (RHS # 87A).
   Outer perianth segment.—color of apex relative to other part Clearly different.
   Outer perianth segment.—color of apex if clearly different from other part Yellow-green.
   Inner perianth segment.—distinct spot in middle of apex Present.
   Inner perianth segment.—color of margin of apex relative to other part Clearly different (Purple).
   Inner perianth segment.—predominant color of exposed part (excluding differently colored margin of apex) Yellow (beige) (RHS # 4D).
   Length of inner perianth segment compared to outer segments.—Longer.
   Extrusion of stamens.—Absent.
   Fragrance.—Present.
   Bulb.—predominant shape Oblate.
   Time of flowering.—Medium.
   Time of flowering.—Long. The variety is not genetically modified and has been tested to be free of Ornithogalum Mosaic Virus.

The invention claimed is:

1. A new and distinct cultivar of Lachenalia named Radient as illustrated and described herein.
